(19) United States
(12) Patent Application Publication
(10) Pub. No.: US 2009/0130694 A1
(43) Pub. Date: May 21, 2009

(54) MAC-2BP as a Marker for the Diagnosis of Gastric Cancer

(76) Inventors: Hee Gu Lee, Daejeon (KR); Yuk Pheel Park, Daejeon (KR); Jae Wha Kim, Daejeon (KR); Eun Young Song, Daejeon (KR); Do Young Yoon, Daejeon (KR); Young Il Yeom, Daejeon (KR); Seung-Chul Choi, Daejeon (KR)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 12/087,224

(22) PCT Filed: Sep. 25, 2006
(86) PCT No.: PCT/KR2006/003802

§ 371 Date: Dec. 29, 2008

(30) Foreign Application Priority Data
Dec. 28, 2005 (KR) 10-2005-0132076

(51) Int. Cl. G01N 33/574

(52) U.S. Cl. 435/7.23

(57) ABSTRACT

Disclosed is a diagnostic kit for gastric cancer. It comprises an agent capable of detecting and quantitatively assaying the gastric cancer marker Mac-2BP (Mac-2 binding protein). Also, a method for detecting the gastric cancer marker using the kit is disclosed.

### TECHNICAL FIELD

The present invention relates to a diagnostic marker for gastric cancer. More particularly, the present invention relates to a diagnostic kit for gastric cancer, comprising an agent capable of detecting Mac-2BP (Mac-2 binding protein), which is identified as a gastric cancer marker. Also, the present invention is concerned with a method for detecting the gastric cancer marker using the kit.

### BACKGROUND ART

Human tumors express and secrete various specific molecules called cancer marker antigens. Currently, a variety of antigens have been provided for the diagnosis and treatment of cancers antigens. As many as 60 tumor markers have been discovered thus far. Among them, some cancer markers are commercially applied, including AFP (hepatic cancer), CEA (colorectal, gastric, pancreatic, breast cancers), HCG (choriocarcinoma), PAP (prostate cancer), NSE (lung cancer), C15-3 (breast cancer), and CA19-9 (colorectal cancer, pancreatic cancer). Gastric cancer is one of the cancers showing the highest morbidity and mortality rates in the world, and is a main cause of death in various countries including Korea, Japan, China, Russia, Hong Kong, and countries in central Europe, Central and South America and Scandinavia. Markers or therapeutic agents which are highly useful in the diagnosis and treatment of gastric cancer, however, have not yet been developed.

Mac-2BP (Mac-2 binding protein) was called at first as a 90 kd. Mac-2BP is a secreted glycoprotein and found in plasma, urine, breast milk and other human fluids, and acts as a ligand binding to galectin-1, -3 and -7. So far, Mac-2BP has been reported to be secreted in a high level amounting to several μg/ml in the plasma of patients with various cancers including breast cancer, lung cancer, colorectal cancer and ovarian cancer. In the case of breast cancer and lung cancer, it has been reported that the level of expression and secretion of Mac-2BP can be used as a marker indicating the metastatic stage and survival rate of patients. However, nowhere is the application of Mac-2BP for the diagnosis of breast cancer mentioned in previous reports.

Leading to the present invention, intensive and thorough research on genes involved in the occurrence and development of cancer cells, conducted by the present inventors, resulted in the finding that a Mac-2BP gene is expressed specifically in gastric cancer cells and that there is a close relationship between the expression and secretion of Mac-2BP and the occurrence of gastric cancer.

### DISCLOSURE OF THE INVENTION

It is therefore an object of the present invention to provide a diagnostic kit for gastric cancer, comprising an agent capable of detecting the gastric cancer marker Mac-2BP.

It is another object of the present invention to provide a method for detecting the gastric cancer marker using the agent.

### BRIEF DESCRIPTION OF THE DRAWINGS

The above and other objects, features and other advantages of the present invention will be more clearly understood from the following detailed description taken in conjunction with the accompanying drawings, in which:

FIG. 1A shows the expression of a Mac-2BP transcript at higher levels in metastatic secondary cancer tissues than in primary cancer tissues with the solid tumor marker Legumain serving as a control, as assayed by Northern blotting. FIG. 1B shows the specific expression of Mac-2BP protein in gastric cancer cell lines as assayed by flow cytometry in which each cell line is fixed with formaldehyde and stained with a monoclonal antibody against Mac-2BP;

FIG. 2A shows the expression levels of Mac-2BP protein in gastric cell lines as assayed by Mac-2BP ELISA using the cell lysates of the cell lines. FIG. 2B shows the levels of Mac-2BP secreted from gastric cell lines SNU-484, -620, and -638, which are different in intracellular expression level, as assayed by ELISA using concentrates of cell culture media;

FIG. 3 shows the overexpression of Mac-2BP at higher levels in gastric cancer tissues (3B and 3D) than in normal tissues (3A and 3C) as assayed by the immunohistochemistry using a monoclonal antibody to Mac-2BP; and

FIG. 4 shows the levels of Mac-2BP secreted to the extracellular environment as assayed by ELISA using sera taken from gastric cancer patients and normal persons.

### BEST MODE FOR CARRYING OUT THE INVENTION

In one aspect, the present invention pertains to a diagnostic kit for gastric cancer, comprising an agent with which the level of mRNA or protein of a Mac-2BP gene can be determined.

In another aspect, the present invention pertains to a method for detecting a gastric cancer marker, comprising the contact of a biological sample with an agent which determines the level of mRNA or protein of a Mac-2BP gene.

The term “marker”, as used herein, means a material that is capable of identifying cancer cells among normal cells, and may be a nucleotide marker for Mac-2BP genes or a polypeptide marker for Mac-2BP proteins. The expression of these makers is increased in gastric cancer cells as compared with the normal cells.

The term “diagnosis”, as used herein, means the identification of the existence or features of gastric cancer and, particularly, is associated with the metastasis of gastric cancer, including both whether or not metastasis has occurred and the possibility of metastasis.

Mac-2BP (NCBI number; L13210) is known to be overexpressed in lung cancer, breast cancer, colorectal cancer and ovarian cancer. For example, the level of Mac-2BP in plasma or other human fluids is recognized as a critical reference indicating the metastasis of cancer cells and the survival rate of patients with lung cancer or breast cancer (Iacobelli S et al., Br. J. Cancer, 1994, 69, 171-76; Antonio M et al., Cancer Res, 2002, 62, 2535-39). However, no publications prior to the present invention disclose the relationship of a Mac-2BP gene with the occurrence of gastric cancer.

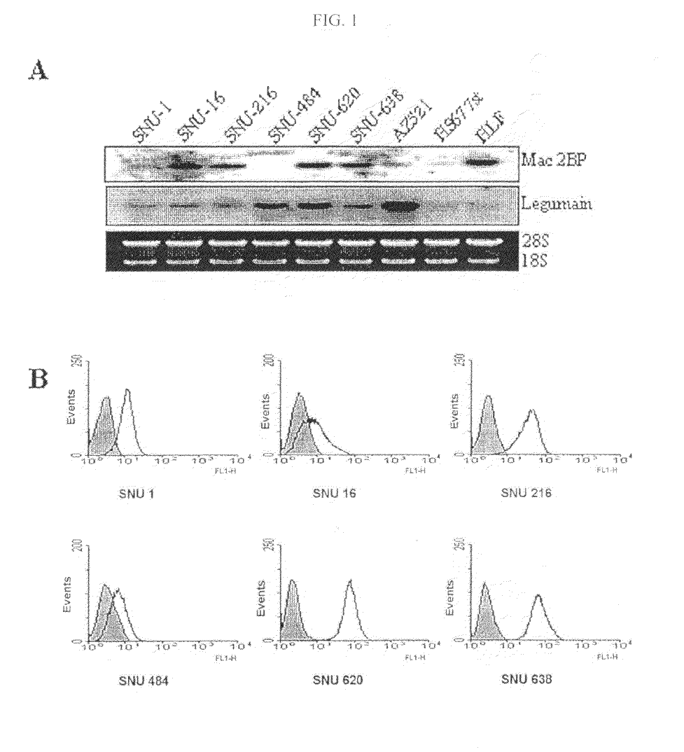

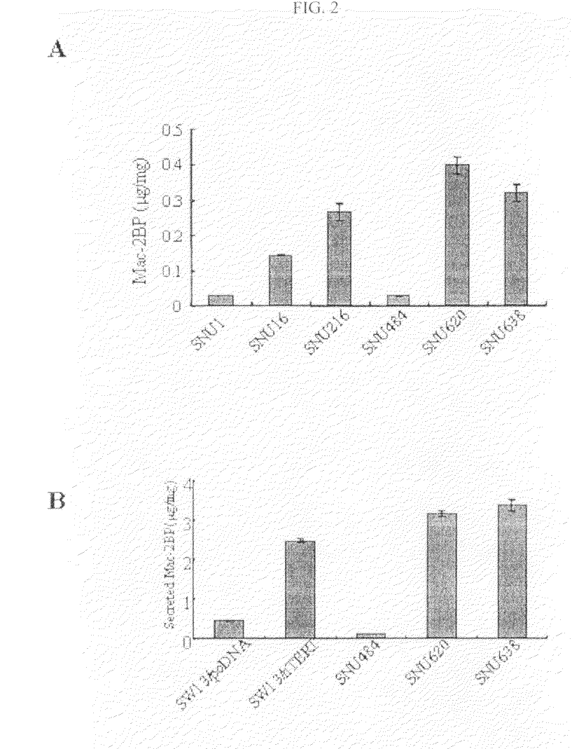

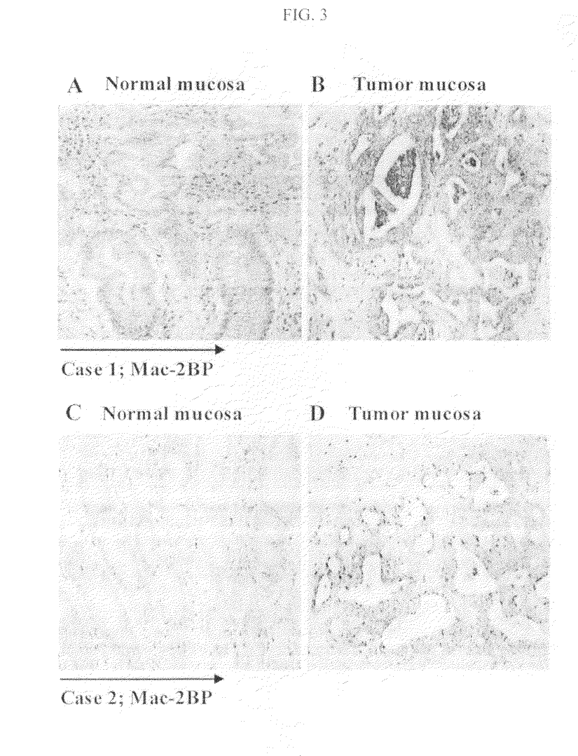

In the present invention, the utility of Mac-2BP as a gastric cancer marker is verified as follows: a Mac-2BP transcript was found to be expressed specifically in the SNU strain, a gastric cancer cell originating in Korean patients. In particular, whereas the primary cancer tissue cells SNU-1 and -484 have very low expression levels of the Mac-2BP transcript, the secondary cancer tissue strains SNU-16, -216, -620 and -638 were distinctively increased in level (FIG. 1A). In this drawing, AZ521 is a gastric cancer cell strain taken from a Japanese patient, and is used as a control. HS677st is a gastric cancer cell strain taken from a Korean patient, and is used as a negative control. HLF (Human lung fibroblast) is obtained from normal human lung tissue. Referring to results in the control cells, Mac-2BP may be well expressed in a normal cell, such as in HLF and may be not expressed in gastric cancer, such as in AZ521. However, the expression of Mac-2BP was found to have high specificity for the Korean gastric cancer cell strain SNU, as compared to the negative control. Legumain, a gene known to be overexpressed in solid cancer cells and to be involved in cancer metastasis, was used as a standard marker (Cheng L et al., Cancer Res. 2003, 63, 2957-2964). A Mac-2BP protein was also examined for specific expression in the gastric cancer cell strains. To this end, intracellular Mac-2BP proteins were stained using a monoclonal antibody. The results of an immunohistochemical staining assay using a monoclonal antibody were similar to those of Northern blotting analysis, demonstrating that Mac-2BP is expressed specifically in SNU-620 and SNU-638, both capable of metastasizing (FIG. 1B). Further, lysates of gastric cancer cell strains were subjected to ELISA in order to quantitatively analyze the expression level of Mac-2BP. Mac-2BP protein was found to be produced at a level of up to 0.4 μg/mg in SNU-620 and -638 each, but was not produced in SNU-484, which has almost no Mac-2BP transcripts therein. These results demonstrate that the level of Mac-2BP transcript is coincident with that of the protein (FIG. 2A). In addition, cell culture media were collected, concentrated with Amicon and analyzed for protein level in order to examine whether Mac-2BP was extracellularly secreted. The extracellular levels of Mac-2BP in the gastric cancer cell strains were about 10 times as much as the intracellular levels thereof. Particularly, SNU-620 and -638 were found to secrete as much as 3 μg/mg of Mac-2BP into the culture media thereof. In contrast, almost no Mac-2BP was detected in the culture medium of SNU-484. Because the gene of Mac-2BP was identified in microarrays using an hTERT-overexpressed clone of the telomerase-negative cell strain SW13, SW13/pcDNA and SW13/hTERT were used as controls in this experiment (FIG. 2B). The demonstration of the fact that the transcript and protein of Mac-2BP gene are specifically expressed in the gastric cancer cells leads to the development of a pharmaceutical composition comprising an inhibitor of the gene of interest, which is useful in the treatment of gastric cancer. In this regard, gastric tissues of patients with gastric cancer were stained using an anti-Mac-2BP monoclonal antibody. Gastric tissues were obtained from 22 individuals, including both normal persons and gastric cancer patients. As many as 70˜75% of the gastric cancer tissues were found to be rich in Mac-2BP. As seen in FIG. 3, Mac-2BP was slightly expressed in normal gastric tissues (A and C), but highly expressed specifically in gastric cancer tissues (B and D). For the quantification of expression of the gene, an s90k/Mac-2BP ELISA kit (Bender Med System GmbH, Austria) was used.

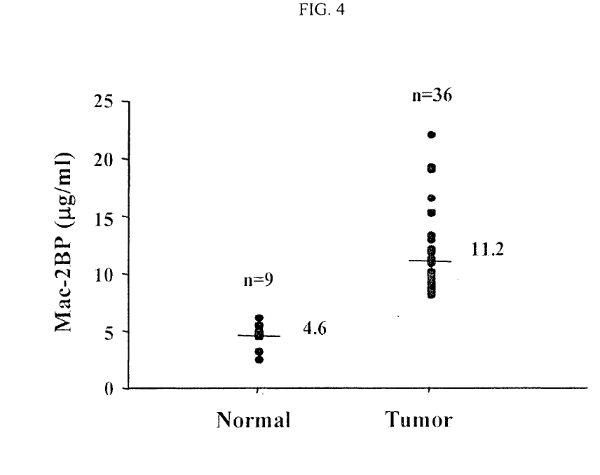

Since the gene of interest is expressed specifically in gastric cancer cells, the plasma of gastric cancer patients was quantitatively analyzed for Mac-2BP through ELISA. Plasma was obtained from 9 normal persons and 36 patients with gastric cancer. The plasma of the patients ranged in Mac-2BP level from 2.4 to 22 μg/ml with an average of 11 μg/ml. This is definitely higher than the average value for normal persons, 4.6 μg/ml, and shows significance with a p value=0.0012 in a Student's t-test (FIG. 4). The relationship between the distribution of plasma Mac-2BP and the clinicopathologic factors of gastric cancer was statistically analyzed. There was no influence of age or sex on the level of Mac-2BP in patient's plasma. However, the expression level of Mac-2BP tends to be higher in a group of patients with secondary metastasis than in a group of patients with no metastasis (p value=0.05). Also, a higher distribution of Mac-2BP was found in a group of patients in TNM clinical stages III and IV than in a group of patients in TNM clinical stages I and II Mac-2BP (p value=0.04), as seen in Table 1, below.

The expression level of the gastric cancer marker gene Mac-2BP in a biological sample can be determined by quantitatively analyzing the mRNA or protein thereof. Various well-known techniques may be used to isolate mRNA and protein from a biological sample and to determine the amounts thereof.

As used herein, the term “biological sample” means a material which allows the analysis of the gastric marker gene Mac-2BP for the level of mRNA or protein thereof, examples of which include tissue, cells, urine, blood, plasma, sera, etc., but are not limited thereto.

By the term “analysis for mRNA level”, as used herein, it is meant that the quantity of mRNA in a biological sample is measured to examine the existence and extent of expression of mRNA of the gastric cancer marker gene Mac-2BP, thereby detecting the gastric cancer marker gene. Analysis methods for mRNA level may be exemplified by RT-PCR, competitive RT-PCR, real-time RT-PCR, RPA (PNase protection assay), Northern blotting, and DNA chip, but are not limited thereto.

Using these methods, the expression level of mRNA can be compared between a normal control and a subject under consideration. Also, these methods are useful to examine whether there is an increase in the level of transcription from the marker gene Mac-2BP to mRNA, which is the basis on which diagnosis of gastric cancer can be conducted.

A kit for analyzing mRNA levels through RT-PCR comprises respective pairs of primers specific for gastric marker Mac-2BP genes. Each of the primers, having a nucleotide sequence specific for a stretch of a marker gene, ranges in length from approximately 7 to 50 bp, and preferably from approximately 10 to 30 bp. The kit may comprise primers specific for a stretch of the nucleotide sequence of a control gene. In addition, the RT-PCR kit may comprises a test tube or a suitable container, a reaction buffer (pH and magnesium concentration variable), dNTPs, Taq-polymerase and reverse transcriptase, DNAse, RNAse-free DEPC-water, and sterile water. The term “primer” means a short nucleotide sequence which can form base pairs with a complimentary template, and has a free 3′ hydroxyl group which serves as a starting point for the DNA replication of the template. A primer is required because most enzymes (e.g., DNA polymerases or reverse transcriptases) that catalyze the replication of DNA can only add to an existing strand of nucleotides under conditions of an appropriate buffer, temperature, various reagents, and four different nucleoside triphosphates. Primers may be incorporated with additional features without changing their fundamental function of serving as a starting point for DNA synthesis. Primers may be chemically synthesized using a phosphoramidite solid support method or some other well-known method. These nucleotide sequences may also be modified through well-known methods.

By the term “analysis for protein level”, as used herein, it is meant that the quantity of the protein encoded by the gastric cancer marker gene Mac-2BP is determined, preferably using an antibody specific therefor so as to examine the existence and extent of expression of the protein.

As used herein, the term “antibody” means a protein molecule which is produced in response to an antigen as part of an immune response. For the purpose of the present invention, the antibody indicates protein molecules coupling specifically to the gastric cancer marker protein Mac-2BP, including polyclonal antibodies, monoclonal antibodies, and recombinant antibodies. Various methods may be used to analyze protein levels using antibodies, examples of which include, but are not limited to, Western blotting, ELISA (enzyme linked immunosorbent assay, RIA (radioimmunoassay), radioimmunodiffusion, ouchterlony immunodiffusion, rocket immunoelectrophoresis, immunohistochemical staining, immunoprecipitation assay, complement fixation assay), FACS, and protein chip.

Using these methods, the quantity of an antigen-antibody complex is compared between a normal control and a subject under consideration. Also, these methods are useful to examine whether there is a significant increase in the expression level from the marker gene Mac-2BP to the protein, which is the basis on which a diagnosis of gastric cancer can be made.

The term “antigen-antibody complex”, as used herein, means a complex which is formed by the binding of an Mac-2BP protein and an antibody specific therefor. The antigen-antibody complex can be quantitatively determined using the signal intensity of a detection label.

Useful in the quantitative analysis of antigen-antibody complex is a detection label selected from among enzymes, fluorescents, ligands, luminescents, microparticles, redox molecules, and radioisotopes. However, other detection labels may be employed in the quantitative analysis of antigen-antibody complex. Examples of the enzyme available as a detection label for the analysis include β-glucuronidase, β-D-glucosidase, β-D-galactosidase, urease, peroxidase, alkaline phosphatase, acetylcholine esterase, glucose oxidase, hexokinase and GDPase, RNase, glucose oxidase and luciferase, phosphofructokinase, phosphoenolpyruvate carboxylase, aspartate aminotransferase, phosphoenolpyruvate decarboxylase, and β-lactamase, but are not limited thereto. As for the fluorescents, they may be exemplified by, but are not limited to, fluorescein, isothiocyanate, rhodamine, phycoerytherin, phycocyanin, allophycocyanin, o-phthaldehyde, and fluorescamine. A biotin derivative may be used as the ligand, but this is non-limiting. Luminescents useful as the detection label include acridinium ester, luciferin, and luciferase, but are not limited thereto. Illustrative, non-limiting examples of the microparticles include colloidal gold and colored latex. The detection label in the form of redox molecules may be ferrocene, ruthenium complexes, viologen, quinone, Ti ions, Cs ions, diimide, 1,4-benzoquinone, hydroquinone, K₄W(CN)₈, [Os (bpy)₃]²⁺, [RU (bpy)₃]²⁺ or [MO(CN)₈]⁴⁻, but is not limited thereto. Useful as radioisotopes are ³H, ¹⁴C, ³²P, ³⁵S, ³⁶Cl, ⁵¹Cr, ⁵⁷ Co, ⁵⁹Fe, ⁹⁰Y, ¹²⁵I, ¹³¹I and ¹⁸⁶Re, but these are not intended to limit the present invention.

The analysis for protein level is preferably conducted using ELISA (enzyme-linked immunosorbent assay). ELISA can be sub-divided into direct sandwich ELISA and indirect sandwich ELISA: in the former, an antigen-antibody complex attached to a solid support is associated with a labeled antibody which can recognize the antigen, and the latter utilizes a labeled secondary antibody which recognizes a primary antibody associated with the antigen of an antigen-antibody complex attached to a solid support. Preferably, the protein level is determined using the sandwich ELISA, in which an antibody attached to a solid support is reacted with a sample to form an antigen-antibody complex and the complex is then associated with a labeled antibody recognizing the antigen, followed by enzyme-mediated color development, or in which a labeled secondary antibody is coupled with an antibody recognizing the antigen of an antigen-antibody complex and is subsequently subjected to enzyme-mediated color development. The level of a complex in which the gastric cancer cell marker protein Mac-2BP is coupled with an antibody can be used to diagnose gastric cancer.

It is also preferable analyze protein level using a protein chip in which one or more antibodies against the gastric cancer marker protein Mac-2BP are arranged and fixed at a high density at predetermined positions on a substrate. In this regard, proteins are separated from a sample and hybridized with a protein chip to form an antigen-antibody complex, which is then read to examine the presence or expression level of the protein of interest, thereby diagnosing the occurrence of gastric cancer.

Another preferred analysis method is a Western blotting technique, which takes advantage of one or more antibodies against the gastric cancer marker Mac-2BP. For Western blotting, proteins are isolated from a sample, separated according to size by electrophoresis, transferred onto a nitrocellulose membrane, and reacted with an antibody against the gastric cancer marker Mac-2BP. The antigen-antibody complex is quantitatively analyzed using a labeled antibody so as to diagnose gastric cancer.

The detection method according to the present invention comprises comparing the expression level of the marker gene between a control affected with no gastric cancer and a cell of interest. The expression level of mRNA or protein may be represented in an absolute quantity of the marker protein (e.g., μg/ml) or in a relative unit (e.g., relative intensity of signal).

In a preferred embodiment, the present invention provides a diagnostic kit for gastric cancer, comprising an antibody binding specifically to a Mac-2BP protein.

In another preferred embodiment, the present invention provides a method for detecting a gastric cancer marker, comprising bringing an antibody against a Mac-2BP protein into contact with a biological sample.

Because Mac-2BP has been identified as a gastric cancer marker, as described above, antibodies thereto can be readily produced using well-known techniques.

Polyclonal antibodies can be prepared using a method well known to those skilled in the art, for example, by injecting the gastric cancer marker protein Mac-2BP into animals and taking the sera from the animals. As a host for producing polyclonal antibodies, any mammalian animal, such as goats, rabbits, sheep, monkeys, horses, pigs, cows, dogs, etc., may be used.

The production of monoclonal antibodies may be conducted using a well-known method, such as a hybridoma method (Köhler and Milstein (1976) European Journal of Immunology 6:511-519), or a phage antibody library technique (Clackson et al, Nature, 352:624-628, 1991; Marks et al, J. Mol. Biol., 222:58, 1-597, 1991).

Typically, a hybridoma method takes advantage of the cells from an immunologically suitable host animal, such as a mouse injected with the antigenic, gastric marker protein Mac-2BP, and a cancer or myeloma cell line. These two kinds of cells are fused with well known method using such as a polyethylene glycol, and the antibody-producing cells thus formed are cultured according to a standard tissue culture method. The positive clones are further subcloned using limited dilution technique to ensure monoclonality. The hybridoma cells which can produce antibodies specific for the gastric cancer marker protein Mac-2BP are cultured in vitro or in vivo on a large scale using a typical technique.

Although they may be used without purification, the monoclonal antibodies produced by the hybridoma cells are preferably purified to a high level using a method well known in the art.

In a phage antibody library method, a large library of phage-displayed human single-chain fragment variable (scFv) antibodies against the gastric cancer marker protein Mac-2BP is constructed in vitro by cloning genes coding for the antibodies and expressing them in the form of fusion proteins on phage surfaces, followed by the separation of monoclonal antibodies binding to the Mac-2BP protein from the library.

For the purification of the antibodies prepared using the above-mentioned methods, various methods may be used, including gel electrophoresis, dialysis, salting out, ion exchange chromatography, and affinity chromatography.

The antibody included in the kit of the present invention may be an intact form consisting of two full-length light chains and two full-length heavy chains or a functional antigen fragment. The term “a functional antigen fragment” means a fragment having an antigenic binding function, exemplified by Fab, F(ab′), F(ab′)2 and Fv.

The kit, comprising an antibody binding specifically to the Mac-2BP protein in accordance with the present invention, is preferably a diagnostic ELISA kit. It may comprise an antibody specific for a control protein, a reagent for detecting an antigen-antibody complex, for example, a labeled secondary antibody, a chromophore, an enzyme (e.g., antibody-conjugated) and a substrate thereof, or other materials binding to the antibody.

A better understanding of the present invention may be obtained through the following examples which are set forth to illustrate, but are not to be construed as the limit of the present invention.

### EXAMPLE 1

### Culturing of Cell Line

SNU-1, -16, -216, -484, -620 and -638, which are Korean gastric cancer cell lines, were purchased from the Korean Cell Line Bank, located at the Medical College of Seoul National University, and cultured in RPMI 1640 (Invitrogen Corporation, Carlsbad, Calif., USA). SW13/pcDNA and SW13/hTERT were cultured in DMEM (Invitrogen Corporation). All of the media used were supplemented with 10% bovine serum (HyClone) and an antibiotic (Life Technologies) before incubation at 37° C. in a 5% CO₂ atmosphere.

### EXAMPLE 2

### Construction of SW13/hTERT Expressing Cell Line

A pcDNA3/hTERT full length plasmid, obtained from the lab of Professor Han-Woong Lee, Ph.D at Sungkyunkwan University, was used as an hTERT cDNA expression vector. On the next day after being seeded at a density of 5×10⁶ cells/100 mm dish, SW13 cells were transformed with the expression vector. For this, 5 mg of the expression vector was diluted in a serum-free medium to form a final volume of 500 ml. Separately, 30 ml of Lipofectamine (GIBCO, Grand Island, N.Y.) was added to 475 ml of a serum-free medium and incubated for 15 min at room temperature. Thereafter, the DNA-added medium was mixed with the Lipofectamine-added medium, followed by incubation at room temperature for 30 min. The cells were washed twice with a serum-free medium. To the DNA-Lipofectamine complex was added to 5 ml of a medium to form a total volume of 6 ml after which the diluted complex was overlaid on the washed cells. The cells were incubated at 37° C. for 6 hrs in a 5% CO₂ incubator before the medium was replaced with a normal growth medium. 24 hrs following the start of transfection, the transformed cells were screened against G418 for two weeks in a medium containing 0.8 mg/ml of G418 (Sigma, St Louis, Mo.). The G418-resistant cells were seeded into 96-well plates at a density of 0.5˜1 cell per well to select monoclones. Finally selected was a SW13/hTERT #31 clone, which exhibited the highest expression rate of hTERT.

### EXAMPLE 3

### RNA Isolation and Northern Blotting

In order to examine whether the Mac-2BP gene was expressed in the gastric cancer cell lines and involved in the progression of gastric cancer, Northern blot analysis was performed. Total RNA was isolated using an acid guanidinium thiocyanate-phenol-chloroform extraction method. For this, first, the cell lines (2˜10×10⁶ cells each) were washed with PBS and lysed with 100 mM 2-mercaptoethanol and 2 M sodium acetate in 4 M guanidine isothiocyanate. Incubation at 4° C. for 1 hr in phenol and chloroform-isoamine alcohol was conducted prior to centrifugation for 30 min. To 600 μl of the supernatant was added an equal volume of isopropanol, followed by incubation at −20° C. for 1 hr or longer. After centrifugation, the RNA pellet thus formed was dried and dissolved in DEPC-treated water. The RNA was analyzed for concentration and purity using a UV spectrophotometer.

The RNA was separated on 2% agarose gel by electrophoresis to identify 18S and 28S bands, and transferred onto a nylon membrane. Hybridization with radiolabeled Mac-2BP probes (NCBI number; L13210, 683 bp-1275 bp) and Legumain probes (NCBI number; NM_{—}005606, 525 bp-1325 bp) was conducted. Exposure to X-ray films for 2 days enabled visualization of the expression of Mac-2BP and Legumain genes in each cell. The results are shown in FIG. 1A.

### EXAMPLE 4

### Analysis of Mac-2BP Expression by Flow Cytometry

Flow cytometry was conducted to examine the intracellular expression level of Mac-2BP. The gastric cancer cell lines were cultured and harvested. These cells were washed once with FACS staining buffer (0.05% BSA, 0.02% sodium azide, in PBS), dissolved in a 2% paraformaldehyde solution, fixed on ice for 15 min, and washed again once with FACS staining buffer. The washed cell pellet was dissolved in a permeabilization buffer (0.1% saponin and 0.05% sodium azide in PBS) and incubated on ice for 15 min. Following washing with FACS staining buffer, the cells were incubated with a Mac-2BP monoclonal antibody (Alexis) for 30 min. Additional washing with FACS staining buffer was conducted prior to incubation with an FITC-conjugated IgG antibody (Molecular Probes) for 30 min on ice. The cells were analyzed with a flow cytometer after being washed three or four times with FACS staining buffer. The results are shown in FIG. 1B.

### EXAMPLE 5

### Quantitative Assay of Mac-2BP by ELISA

Each cell line was harvested and completely lysed in a lysis buffer (0.01% Nonidet P-40, 10 mM Tris, pH 7.6, 50 mM KCl, 5 mM MgCl₂, 2 mM dithiothreitol, 20% glycerol+protease inhibitor cocktail) (Sigma) for 40 min. Water-insoluble proteins were removed by centrifugation at 12,000 rpm at 20 min before quantitative protein analysis. The same amount of protein was used in Mac-2BP ELISA. For this, an s90k/Mac-2BP ELISA kit (Bender Med System) was used. The protein mixture was incubated overnight with Mac-2BP monoclonal antibody-coated strips at 4° C. to attach the Mac-2BP thereto, followed by washing four times with a washing buffer. Then, the strips were incubated with an HRP-conjugated secondary antibody at 37° C. for 1 hr, washed four times with a washing buffer, and incubated in 100 ml of a substrate solution for 5 min to develop color before reaction termination with a stop buffer. The well plates were introduced into an ELISA reader, and the absorbance was read at 540 nm and analyzed using a SoftMax program.

When using cell cultures, proteins were concentrated and dialyzed before ELISA analysis for Mac-2BP. As for sera from gastric cancer patients, they were diluted 1:200 before ELISA analysis for Mac-2BP. The results are shown in FIGS. 2A, 2B and 4.

### EXAMPLE 6

### Collection of Culture Media for Gastric Cancer Cell Lines and SW13 Cell Line

In order to quantitatively analyze extracellularly secreted Mac-2BP, culture media for the gastric cancer cell lines and SW13 cell line were collected. After being cultured in 15 cm-culture dishes, each cell line was washed with, and then incubated in, a serum-free medium. After 24 hours of incubation, the cell cultures were centrifuged twice at 2,000 rpm for 10 min to collect media free of cells. The media were concentrated using Amicon (cutoff MW 50,000 Da, Millipore). The concentrates were placed in active dialysis bags and dialyzed three or more times against 1×PBS at 4° C. at intervals of 4 hrs. The concentrations of proteins were determined using a BioRad protein assay kit before use in ELISA.

### EXAMPLE 7

### Detection of Mac-2BP Protein by Immunohistochemical Staining

Immunohistochemical staining was conducted in order to examine the expression level of Mac-2BP in gastric cancer tissues. Paraffin sections in which gastric cancer tissues taken from gastric cancer patients were embedded were deparaffinized with xylene and hydrated using graded alcohol washes. The hydrated sections were placed in 10 mM citric acid buffer (pH 6.0) after which microwaves were radiated three times to the hydrated sections for 5 min. Endogenous peroxidase was blocked by treatment with 3% hydrogen peroxide in methanol for 6 min. Thereafter, the sections were treated for 30 min with a working solution of a Vector kit (Cat No. PK6102) so as to prevent non-specific protein binding. Incubation was carried out with a Mac-2BP IgG antibody diluted in 1×PBS at room temperature for 2 hrs and then with biotinylated anti-mouse Ig Ab (Vector kit) at room temperature for 30 min. Afterwards, the sections were treated with an ABC Elite kit (Vector kit) at room temperature for 30 min and then reacted with a diaminobenzidine tetrahydrochloride substrate (Vector kit) for 2 min to analyze the expression level of Mac-2BP in gastric cancer tissues (FIG. 3). As seen in FIG. 3, Mac-2BP was expressed at distinctively high levels in the gastrointestinal tissues affected with cancer (FIGS. 3B and 3D), but at very low levels in normal gastrointestinal tissues (FIGS. 3A and 3C).

### EXAMPLE 8

### Relationship Between Distribution of Plasma Mac-2BP and Clinicopathologic Factors

The relationship between the distribution of plasma Mac-2BP and the clinicopathologic factors of gastric cancer, shown in FIG. 4, was statistically analyzed and is summarized in Table 1, below.

TABLE 1

Mac-2BP Levels

| Characteristics | n | High (%)^{a} | p value^{b} |
|---|---|---|---|
| All | 36 | 14(39) | |

Age (mean)

| | | | |
|---|---|---|---|
| =61.5 years | 19 | 7(37) | |
| >61.5 years | 17 | 7(41) | 0.28 |

Gender

| | | | |
|---|---|---|---|
| Male | 24 | 9(37) | |
| Female | 12 | 5(42) | 0.27 |

Distant metastasis

| | | | |
|---|---|---|---|
| Present | 25 | 12(48) | |
| Absent | 11 | 2(18) | 0.05 |

Clinical stage

| | | | |
|---|---|---|---|
| I + II | 18 | 6(33) | |
| III + IV | 18 | 8(44) | 0.04 |

^{a}Number (percentage) of gastric cancer patients with Mac-2BP levels above the cutoff value of 11.2 g/ml.
^{b}p-values were determined by Mann-Whitney U test.

As is apparent from the data of Table 1, the level of Mac-2BP in patient's plasma was not influenced by age or sex. However, the expression level of Mac-2BP tends to be higher in a group of patients with secondary metastasis than in a group of patients with no metastasis (p value=0.05). Also, a higher distribution of Mac-2BP expression was found in a group of patients in TMN clinical stages III and IV than in a group of patients in TMN clinical stages I and II (p value=0.04).

### INDUSTRIAL APPLICABILITY

As described hitherto, the Mac-2BP gene is overexpressed specifically in gastric cancer cells and secreted into the extracellular environment of cells. In addition, the gene of interest is expressed at distinctively high levels in the gastrointestinal tissues of cancer cell tissues having metastatic capacity. Therefore, gastric cancer, particularly metastasizing or metastatic gastric cancer can be diagnosed by detecting the level of mRNA or protein of the Mac-2BP gene. Plasma was found to have a Mac-2BP level of 11 μg/ml on average over 36 patients with gastric cancer, which is definitely higher than the average value for 9 normal persons, 4.6 μg/ml. Antibodies binding specifically to Mac-2BP are useful in the diagnosis of gastric cancer.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A diagnostic kit for gastric cancer, comprising an antibody binding specifically to Mac-2BP (Mac-2 binding protein).

2. The diagnostic kit according to claim 1, wherein the gastric cancer is metastatic or capable of metastasizing.

3. The diagnostic kit according to claim 1, wherein the diagnostic kit allows protein levels to be compared between a control and a sample of interest.

4. The diagnostic kit according to one of claim 1, wherein the diagnostic kit utilizes an ELISA (Enzyme-linked immunosorbent assay) method.

5. The diagnostic kit according to claim 4, wherein the diagnostic kit utilizes a sandwich ELISA method.

6. A method for detecting a gastric cancer marker, comprising contacting an antibody binding specifically to a Mac-2BP protein with a biological sample selected from among urine, blood, serum, and plasma.

7. The method according to 6, wherein the gastric cancer marker is metastatic or capable of metastasizing.

8. The method according to 6, wherein the method allows protein levels to be compared between a control and a sample of interest.

9. The method according to claim 6, wherein the method utilizes an ELISA (Enzyme-linked immunosorbent assay) method.

10. The method according to 9, comprising:
(a) providing a biological sample of interest;
(b) contacting the sample with an antibody binding specifically to Mac-2BP protein;
(c) quantitatively analyzing an antigen-antibody complex; and
(d) comparing the quantitative analysis result of step (c) with that of a normal control.
